(19) United States
(12) Patent Application Publication
(10) Pub. No.: US 2024/0350698 A1
(43) Pub. Date: Oct. 24, 2024

(54) TRANSPARENT DISINFECTING LIGHTING DEVICE

(71) Applicant: SIGNIFY HOLDING B.V., EINDHOVEN (NL)

(72) Inventors: MICHEL CORNELIS JOSEPHUS MARIE VISSENBERG, ROERMOND (NL); JOHANNES PETRUS MARIA ANSEMS, HULSEL (NL)

(21) Appl. No.: 18/285,878

(22) PCT Filed: Apr. 1, 2022
(86) PCT No.: PCT/EP2022/058773

§ 371 Date: Oct. 6, 2023

(30) Foreign Application Priority Data
Apr. 6, 2021 (EP) 21166959.3

(51) Int. Cl. A61L 9/20; F21V 7/28; F21V 33/00; F21Y 115/10; G02B 27/30

(52) U.S. Cl. CPC A61L 9/20; F21V 7/28; F21V 33/0064; F21V 33/0096; G02B 27/30; A61L 2209/12; F21Y 2115/10

(57) ABSTRACT

The present invention relates to a disinfecting lighting device (1) comprising a housing (2) comprising an air disinfection cavity (3) delimited by a wall element (4). The wall element (4) comprises at least one transparent portion (5, 5′) being substantially transparent to light from at least a portion of visible wavelength range. The disinfecting lighting device (1) further comprises at least one disinfecting light source (6) arranged inside 5the air disinfection cavity (3) and providing disinfecting light. The disinfecting lighting device (1) comprises at least one air inlet duct (7) and at least one air outlet duct (8) arranged in the wall element (4). The at least one transparent portion (5, 5′) comprises a reflective coating (9) for reflecting the disinfecting light.

### TECHNICAL FIELD

The present invention relates to a disinfecting lighting device having improved appearance and function. The present invention further relates to an air disinfecting system comprising such a disinfecting lighting device.

### BACKGROUND

In view of the recent development in the world concerning the global COVID-19 pandemic, disinfection has become a topic of renewed interest as the demand for sterilization increases. One way of disinfecting involves the use of UV light. As a response to pathogenic outbreaks involving airborne microorganisms it would be beneficial to employ UV light for disinfecting air and objects at locations where the transmission of such microorganisms is believed to occur, i.e. in spaces with high density of humans, such as offices, department stores, pharmacies, hospitals and public transport.

Currently existing disinfecting luminaires are used to flood spaces such as a hospital rooms with UV-B (ultra-violet light of 280-315 nanometer (nm)) and UV-C (ultra-violet light of 200-280 nm) radiation for disinfection purposes. Such disinfecting luminaires require a relatively brief time, e.g. several minutes, to achieve adequate disinfection but require the room to be evacuated of people. Another type of disinfecting luminaire uses a fixed 405 nm violet light source to provide disinfection without evacuating people from the room. However, such luminaires may require hours to achieve adequate disinfection because their light is less effective at killing pathogens than UV-B and UV-C radiation and is dispersed over a wide area so that the irradiance level is relatively low.

The increased demand for germicidal activities may involve operating disinfecting light sources in environments with human presence, thus introducing a risk for unintentional irradiation by UV light. Therefore, disinfecting light sources, in particular those involving UV light, should possess reliable safety features in order to avoid potential exposure of humans or animals to the harmful irradiation.

One of the current solutions for increasing safety of disinfecting lighting devices is enclosing the UV light source inside a unit such as a box or an air vent from which the UV light cannot escape thus causing damage to living species. Typically, forced ventilation through the unit is needed to effectively purify the air in a room. Such units are normally attached to a ceiling or a wall leading to rather low efficiency. Placement of such units in the middle of a room in order to ameliorate the air flow through the unit thus increasing efficiency is inappropriate since the currently existing units are bulky and obstruct the view.

Several attempts have been made to improve disinfecting lighting devices of the prior art. For instance, CN201244218 discloses an ultraviolet light sterilizing device with function of illumination. The sterilizing device comprises a framework, wherein a silver reflection layer is coated on the internal surface of the framework, and a plurality of air inlets and air outlets are arranged on the lateral side of the framework for the air to circulate in the framework. A UV lamp is arranged in the framework and can sterilize the air in the framework, and an optical board is arranged on a light outlet surface of the framework and can transfer the UV light emitted from the UV lamp into visible light for illumination. The sterilizing device of CN201244218 is safe since the UV light is converted into visible light before leaving the device. However, the device of CN201244218 suffers from disadvantage of rather low disinfecting efficiency since the UV light is absorbed and converted. In view of the above, it is desired to obtain a disinfecting lighting device having a high level of safety and improved efficiency compared to the solutions suggested by the prior art.

### SUMMARY

The present invention thus provides such a disinfecting lighting device. The disinfecting lighting device of the present invention may be particularly suitable for disinfecting spaces with high level of activity, such as a waiting room in a hospital or a veterinary clinic, a public space such as a library, an office, a department store or the like, as well as public transportation means, such as busses or trains.

The present invention provides a disinfecting lighting device comprising:
a housing comprising an air disinfection cavity delimited by a wall element, the wall element comprising at least one transparent portion being substantially transparent to light from at least a portion of visible wavelength range;
at least one disinfecting light source arranged inside the air disinfection cavity and providing disinfecting light;
at least one air inlet duct and at least one air outlet duct arranged in the wall element;
wherein the at least one transparent portion comprises a reflective coating for reflecting the disinfecting light.

The disinfecting lighting device according to the present invention is therefore extremely safe since the disinfecting light will be kept inside the air disinfection cavity by the reflective coating. Further, the disinfecting lighting device of the present invention has improved efficiency, since the disinfecting light is reflected from the transparent portion into the air disinfection cavity, minimizing irradiation losses. Finally, the disinfecting lighting device is aesthetically appealing.

The housing of the disinfecting lighting device may be of any suitable shape and size. In particular, the housing may be a sphere, a hemisphere, a cylinder, a pyramid, or a prism. By the term “wall element” is understood the entire surface of the housing. The wall element has longitudinal extension, herein being understood as circumference of the wall element, and a transverse extension, herein being understood as thickness of the wall element. The wall element in the context of the present invention may comprise a plurality of flat surfaces being arranged at an angle relatively each other. By the term “plurality” in the context of the present invention is meant two or more.

Preferably, the housing is a cuboid comprising six substantially flat rectangular faces, wherein each face is perpendicular to the adjacent faces, and is parallel to the non-adjacent face. Further, two parallelly arranged faces may have an area being significantly larger than the area of the remaining faces. Such faces will hereinafter be referred to as “main faces”. In such an embodiment the housing is thin and flat, such that it may be arranged in any location in a room without creating an obstacle for people that move around and without being perceived as an aesthetically disturbing element.

The air disinfection cavity according to the present invention is delimited by the wall element. Therefore, the air disinfection cavity may be understood as the inner space of the housing. The shape of the air disinfection cavity will thus depend on the shape of the housing. The air disinfection cavity may comprise a central portion, herein defined as the portion being farthest away from the wall element, and the peripheral portion, herein defined as the portion being closest to the wall element.

According to the present invention, the wall element comprises at least one transparent portion being substantially transparent to light from at least a portion of visible wavelength range. By the term “visible wavelength range” is understood light having wavelength from 380 nm to 750 nm. The at least one transparent portion may be substantially transparent to light from at least a portion of the wavelength range from 485 nm to 750 nm. In other words, the transparent portion may be transparent to green, yellow, and red light. Such an embodiment may be useful since violet and blue light may be used for disinfection and are known to be harmful at high intensity. Preferably, the transparent portion according to the present invention is transparent to light from the entire visible wavelength range.

The size and placement of the transparent portion may depend on the shape of the housing and on the required design. For instance, when the housing is in the form of a sphere, the transparent portion may be a hemisphere. Such a disinfecting lighting device may be used a decorative suspended design element in a room, while having disinfecting function.

Another particularly preferred embodiment is the housing in the form of a thin and flat cuboid described above, wherein each of the main faces constitutes the transparent portion. The disinfecting lighting device according to such an embodiment may be arranged on an attachment surface such that the two parallelly arranged transparent portions are perpendicular to the attachment surface. The disinfecting lighting device according to such an embodiment is thin, flat, and completely transparent to visible light, thus being particularly useful as a counter shield, desk divider, room divider or the like. The disinfecting lighting device having thin, flat, and transparent housing will not obstruct sound and view and will at the same time have germicidal effect on the air passing though the disinfecting lighting device, thus preventing spread of microbial species.

The transparent portion may be manufactured of a transparent material such as PMMA or glass. If a plurality of transparent portions is present, the transparent portions may be manufactured of the same material or of different materials. Preferably, the at least one transparent portion is UV-absorbing. Such a property may be achieved by manufacturing the transparent portion of a UV-absorbing material, or providing the material of the transparent portion with a UV-absorbing additive.

The disinfecting lighting device according to the present invention comprises at least one disinfecting light source arranged inside the air disinfection cavity and providing disinfecting light.

The disinfecting light source may be any light source configured to emit light having a high germicidal effect. In particular, the light emitted from the disinfecting light source may be within the UV-spectrum. In the context of the present invention, the UV-spectrum comprises any electromagnetic radiation with a wavelength from 10 nm to 400 nm. Further, the disinfecting light source may emit at least one of: UV-C radiation (100 nm-280) nm), UV-B radiation (280 nm-315 nm), UV-A radiation (315 nm-400 nm), violet light, and blue light.

The disinfecting light source may be a UV gas discharge lamp, solid-state light source such as a light-emitting diode, LED, and/or a laser diode. Further, the disinfecting light source may be a low pressure mercury plasma lamp or an excimer light source. The disinfecting light source may comprise a plurality of light emitting diodes (LEDs) each of which emits at least one of: UV-C radiation (100 nm-280 nm), UV-B radiation (280 nm-315 nm), UV-A radiation (315 nm-400 nm), violet light, and blue light.

The term “LED” as used in the context of the present invention implies any type of LED known in the art, such as inorganic LED(s), organic LED(s), polymer/polymeric LEDs, violet LEDs, blue LEDs, optically pumped phosphor coated LEDs, optically pumped nano-crystal LEDs. As used herein, the term “LED” can encompass a bare LED die arranged in a housing, which may be referred to as a LED package. When UV-C light is used, the LED may be mounted in a cavity covered in a non-contact manner by an emission window made from quartz/fused silica.

The plurality of LEDs may comprise at least 10 LEDs, preferably at least 20 LEDs, more preferably at least 30 LEDs.

The disinfecting lighting device according to the present invention may comprise a plurality of disinfecting light sources, each providing disinfecting light. The wavelengths of the light emitted from each disinfecting light source may be same or different. For example, the disinfecting lighting device may comprise three disinfecting light sources, wherein the first one of the disinfecting light sources emits light within the UV-C spectrum, the second one of the disinfecting light sources emits light within the UV-B spectrum, and the third one of the disinfecting light sources emits light within the UV-A spectrum. The various light sources might be used together or might be activated selectively depending on the type of microbiological species that needs to be deactivated.

The disinfecting light source may be arranged in the peripheral portion of the air disinfection cavity or in the central portion of the air disinfection cavity. In particular, the disinfecting lighting device may comprise two disinfecting light sources being arranged opposite to each other in the peripheral portion of the air disinfection cavity.

In order to enable flow of air to be purified through the disinfecting lighting device, the disinfecting lighting device according to the present invention comprises at least one air inlet duct and at least one air outlet duct arranged in the wall element through the entire transverse extension of the wall element. The air enters the disinfecting lighting device through the air inlet duct into the air disinfection cavity, wherein the germicidal disinfecting light emitted by the disinfecting light source deactivates microbial species, such as virus and bacteria. The purified air is than allowed to exit the disinfecting lighting device through the air outlet duct. The flow of air may be created by means of any device known in the art, such as a fan, a HVAC system or forced ventilation, or may be enabled by natural convection. When the term “air ducts” is used hereinbelow, it means both the at least one air inlet duct and at least one air outlet duct.

The placement of the at least one air inlet duct and the at least one air outlet duct may be at any location along the wall element of the disinfecting lighting device. Preferably, the at least one air inlet and the at least one air outlet are positioned on the opposite sides of the wall element in order to maximize the residing time of the air inside the air disinfection cavity. The disinfecting lighting device may comprise a plurality of the air inlet ducts and/or a plurality of the air outlet ducts, such that the flow of air is facilitated through the air disinfection cavity.

The at least one air inlet duct and the at least one air outlet duct may have any suitable cross-section, such as circular, oval, rectangular, square or the like. The cross-section of the at least one air inlet duct may be same as or different from the cross-section of the at least one air outlet duct. If only one air inlet duct and only one air outlet duct are present, the air ducts may be in the form of elongated slits. Further, if a plurality of air inlet ducts is present, the cross-section of each air inlet duct in the plurality of the air inlet ducts may be same as or different from the cross-section of the other air inlet ducts. The same is valid for the air outlet ducts.

The cross-section area of the at least one air inlet duct and/or the at least one air outlet duct may be of any suitable size. Generally, the cross-section area depends on the shape of the air ducts, thickness of the wall element, and the number of ducts. If only one air inlet duct and only one air outlet duct are present, the cross-section area may be from 10 cm² to 100 cm². In a plurality of air inlet ducts and air outlet ducts are present, the cross-section area of the at least one air inlet duct and/or the at least one air outlet duct may be from 1 mm² to 1 cm². The cross-section area of the at least one air inlet duct may be same as or different from the cross-section area of the at least one air outlet duct. Further, if a plurality of air inlet ducts is present, the cross-section area of each air inlet duct in the plurality of the air inlet ducts may be same as or different from the cross-section area of the other air inlet ducts. The same is valid for the air outlet ducts.

The at least one air inlet duct and the at least one air outlet duct should be arranged such that the disinfecting light emitted by the disinfecting light source is prevented from escaping the air disinfection cavity though the air ducts. The at least one air inlet duct and/or the at least one air outlet duct may be arranged perpendicularly to the longitudinal extension of the wall element. Alternatively, the at least one air inlet duct and/or the at least one air outlet duct may be angled relatively the longitudinal extension of the wall element such that the disinfecting light is prevented from passing through the at least one of the air inlet duct and/or the at least one air outlet duct. The angle between the at least one air inlet duct and/or at least one air outlet duct and the wall element may be from 20° to 90°. Further, if a plurality of air inlet ducts is present, the angle between each air inlet duct in the plurality of the air inlet ducts and the wall element may be same as or different from the angle between the other air inlet ducts and the wall element. The same is valid for the air outlet ducts.

The at least one air inlet duct and/or the at least one air outlet duct may be arranged in the at least one transparent portion. Further, the at least one transparent portion may comprise a first layer and a second layer, wherein each of the first layer and the second layer comprises at least one air inlet duct and at least one air outlet duct. The first and the second layer may then be arranged adjacent to each other forming an intermediate air duct, such that the at least one air inlet duct in the first layer is offset relative the at least one air inlet duct in the second layer, and the at least one air outlet duct in the first layer is offset relative the at least one air outlet duct in the second layer. The air ducts according to such an embodiment may be perpendicular or angled in relation to the wall element and/or to the intermediate air duct. The air to be disinfected will thus pass through the at least one air inlet duct in the first layer of the transparent portion, move through the intermediate air duct and continue to the air disinfection cavity through the at least one air inlet duct in the second layer of the transparent portion. When the purified air exits the air disinfection cavity, it is first directed through the at least one air outlet duct in the second layer of the transparent portion, moves through the intermediate air duct and exits to the ambient through the at least one air outlet duct in the first layer of the transparent portion.

It is further conceivable to arrange the first and the second layer of the transparent portion in direct contact with each other, i.e. without formation of an intermediate air duct. In this case, the air ducts in the first layer of the transparent portion should be aligned with the air ducts in the second layer in order to allow flow of air through the layers. In order to prevent the disinfecting light from escaping the air disinfection cavity, the air ducts in at least one of the first layer and the second layer should be angled in relation to the wall element.

In order to prevent the disinfecting light from escaping the air disinfection cavity, the at least one transparent portion comprises a reflective coating for reflecting the disinfecting light. The reflective coating should completely cover the transparent portion. If a plurality of transparent portions is present, each of the transparent portion comprises the reflective coating.

Preferably, the reflective coating is arranged on the inner side of the transparent portion, i.e. on the side facing the air disinfection cavity. According to such an embodiment, the disinfecting light will be reflected before it hits the transparent portion. Therefore, interaction of the disinfecting light with the material of the transparent portion will be minimized or eliminated, thus preventing the material from degradation, and prolonging the service life of the disinfecting lighting device.

The reflective coating according to the present invention may comprise a plurality of layers, wherein at least one of the plurality of layers may comprise HfO₂—SiO₂. Further, at least one of the plurality of layers may comprise quartz materials such as Al₂O₃, MgF₂ or mixtures thereof. The reflective coating may be in the form of an interference stack, also referred to as a dichroic filter. Such an embodiment is based on multiple thin layers, typically thinner than the wavelength of the light it acts upon. The number of layers as well as thickness of each layer may be optimized to reflect light having a certain wavelength range and a certain range of incoming angles. The reflective coating should be transparent to light from at least a portion of the visible wavelength range.

The lighting device of the present invention may comprise at least one collimating optical element arranged for collimating the disinfecting light in a direction being parallel to the longitudinal extension of the at least one transparent portion. According to such an embodiment, interactions between the disinfecting light and the transparent portion is minimized or eliminated. Further, the angle of incidence of the disinfecting light on the transparent portion is always large, which facilitates engineering of the multilayer reflective coating for optimal reflection of the disinfecting light. Finally, absorption losses of the disinfecting light per reflection are minimized.

The disinfecting lighting device of the present invention may further comprise at least one non-disinfecting light source arranged to emit a non-disinfecting light. By the term “non-disinfecting light” in the context of the present invention is understood as light having no or less efficient germicidal properties than the disinfecting light. The non-disinfecting light source may be adapted for, in operation, emitting visible light, such as for instance white light. Thus, the non-disinfecting light may be visible light, in particular white light.

The disinfecting light source and the non-disinfecting light source may be arranged as a single light source, such as e.g. a lamp such as a TL tube or a LED TL tube.

Alternatively, the non-disinfecting light source may be a separate light source and may be arranged next to the disinfecting light source. The non-disinfecting light source may be used for providing a visual alert signal to the user once the disinfecting light source is operating, e.g. by starting to emit visible light of different colour, in particular red colour, or by flashing the visible light.

Further, the non-disinfecting light source may be used for general illumination, for creating privacy window; for providing decorative colours, for emitting enhanced desk lighting or combinations thereof.

The non-disinfecting light source adapted for, in operation, emitting visible light may for instance be provided as phosphor converted UV LEDs and/or blue LEDs or as RGB LEDs. The non-disinfecting light source may comprise a plurality of LEDs that emit white light.

The at least one non-disinfecting light source may be arranged adjacent to the at least one transparent portion. In particular, the at least one non-disinfecting light source may be embedded between the transparent portion and the wall element. In such an embodiment, the transparent portion comprises at least one light extraction element arranged for extraction of the non-disinfecting light. Such an embodiment is advantageous since the non-disinfecting light source is hidden from view, thus providing pleasant lighting conditions.

The at least one non-disinfecting light source may be electrically coupled to the disinfecting light source. In such an embodiment, the user will be automatically notified that the disinfecting light source is operating.

The present invention further relates to an air disinfecting system comprising a circulation means for generating a flow of air in a room, the flow of air being directed in a flow direction. The air disinfecting system further comprises the disinfecting lighting device according to any one of the embodiments described above. The at least one air inlet duct and/or at least one air outlet duct is arranged substantially parallel to the flow direction. Thus, the flow of air will be forced to enter the air disinfection cavity of the disinfecting lighting device through the at least one air inlet, wherein it will be purified and released through the at least one air outlet. The circulation means may be a fan or HVAC system.

The disinfecting lighting device of the present invention may be a luminaire.

The disinfecting lighting device may be configured to suspend from a ceiling of a room by a suspension arrangement or may be arranged at any other surface within the room, such as on a wall, on the floor or on a surface of a piece of furniture.

Considering the above, the present invention provides a highly efficient disinfecting lighting device and an air disinfecting system comprising such a lighting device. The disinfecting lighting device provides high safety level, allowing ongoing disinfection even when humans and/or animals are present in the area that is being disinfected. Moreover, the safety level and the appearance of the disinfecting lighting device allows positioning the device anywhere in the room, such as a wall, a table or the floor, which in turn provides an efficient disinfection.

### BRIEF DESCRIPTION OF THE DRAWINGS

Embodiments of the invention will now be described by way of example with reference to the accompanying drawings, of which:

FIG. 1 illustrates a disinfecting lighting device according to the present invention:

FIG. 2 depicts a disinfecting lighting device comprising two disinfecting light sources:

FIG. 3 illustrates a disinfecting lighting device comprising a collimating optical element:

FIG. 4 depicts a disinfecting lighting device comprising layered transparent portions:

FIGS. 5a-5c illustrate various embodiments of the layered transparent portion:

FIGS. 6 and 7 show a disinfecting lighting device comprising two non-disinfecting light sources:

FIG. 8 illustrates an air disinfecting system according to the present invention:

FIG. 9 depicts a disinfecting lighting device arranged on a wall:

FIG. 10 shows a disinfecting lighting device being used as a desk divider;

FIG. 11 illustrates a disinfecting lighting device being used as a counter shield.

All the figures are schematic, not necessarily to scale, and generally only show parts which are necessary in order to elucidate embodiments of the present invention, wherein other parts may be omitted or merely suggested.

### DETAILED DESCRIPTION

The present invention will now be described hereinafter with reference to the accompanying drawings, in which exemplifying embodiments of the present invention are shown. The present invention may, however, be embodied in many different forms and should not be construed as limited to the embodiments of the present invention set forth herein; rather, these embodiments of the present invention are provided by way of example so that this disclosure will convey the scope of the invention to those skilled in the art. In the drawings, identical or similar reference numerals denote the same or similar components having a same or similar function, unless specifically stated otherwise.

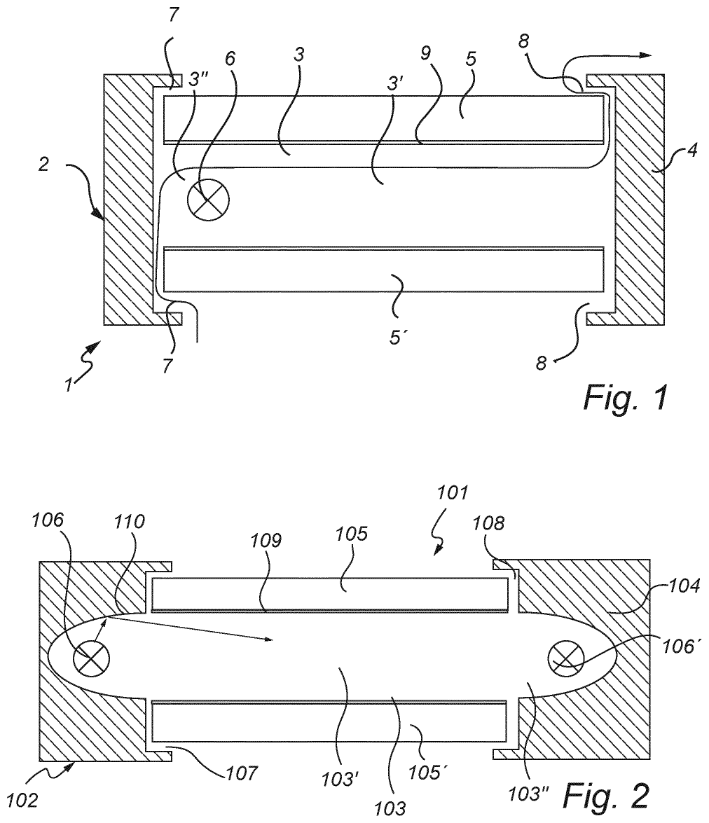

FIG. 1 illustrates a disinfecting lighting device 1 according to the present invention. The disinfecting lighting device 1 comprises a housing 2 comprising an air disinfection cavity 3 delimited by a wall element 4. Therefore, the air disinfection cavity 3 may be understood as the inner space of the housing 2. The air disinfection cavity 3 comprises a central portion 3′ and a peripheral portion 3″.

The wall element 4 comprises two transparent portions 5 and 5′ being substantially transparent to light from visible wavelength range and UV absorbent. The disinfecting lighting device 1 comprises one disinfecting light source 6 arranged inside the air disinfection cavity 3 and providing disinfecting UV light. The disinfecting light source 6 is arranged in the peripheral portion 3″ of the air disinfection cavity 3. As may be seen in FIG. 1, the transparent portions 5 and 5′ comprise a reflective coating 9 for reflecting the disinfecting light.

As mentioned above, the disinfecting lighting device 1 according to the present invention is therefore extremely efficient since the disinfecting light will be kept inside the air disinfection cavity 3 by the reflective coating 9. Further, the disinfecting lighting device 1 of the present invention is safe because the transparent portions are absorbing for UV and only transparent for visible light. Further, the disinfecting lighting device is efficient since the reflective coating reflects the disinfecting light from the transparent portions 5, 5′ into the air disinfection cavity 3, minimizing irradiation losses. Finally, the disinfecting lighting device 1 is aesthetically appealing since the transparent portions 5, 5′ are transparent for visible light.

The housing 2 of the disinfecting lighting device 1 is a cuboid comprising six substantially flat rectangular faces, wherein each face is perpendicular to the adjacent faces, and is parallel to the non-adjacent face. Further, two parallelly arranged faces 5, 5′ have an area being significantly larger than the area of the remaining faces.

In order to enable flow of air to be purified through the disinfecting lighting device 1, the disinfecting lighting device 1 comprises two air inlet ducts 7 and two air outlet ducts 8 arranged in the wall element 4 through the entire transverse extension of the wall element 4. The air enters the disinfecting lighting device 1 through the air inlet ducts 7 into the air disinfection cavity 3, wherein the disinfecting UV light emitted by the disinfecting light source 6 deactivates microbial species, such as virus and bacteria. The purified air is than allowed to exit the disinfecting lighting device 1 through the air outlet ducts 8.

The air inlet ducts 7 and the air outlet ducts 8 are positioned on the opposite sides of the wall element 2 in order to maximize the residing time of the air inside the air disinfection cavity 3.

As may be seen in FIG. 1, the air inlet ducts 7 and the air outlet ducts 8 are arranged such that the disinfecting light emitted by the disinfecting light source 6 is prevented from escaping the air disinfection cavity 3 though the air inlet ducts 7 or the air outlet ducts 8 by introduction of a 90° turn in the air inlet ducts 7 and in the air outlet ducts 8.

In order to prevent the disinfecting light from escaping the air disinfection cavity 3, the transparent portions 5, 5′ comprise a reflective coating 9 for reflecting the disinfecting light. The reflective coating 9 completely covers the transparent portions 5 and 5′. The reflective coating 9 is arranged on the inner side of the transparent portions 5, 5′, i.e. on the side facing the air disinfection cavity 3. As mentioned above, according to such an embodiment, the disinfecting light will be reflected before it hits the transparent portions 5, 5′. Therefore, interaction of the disinfecting light with the material of the transparent portions 5, 5′ will be minimized or eliminated, thus preventing the material form degradation, and prolonging the service life of the disinfecting lighting device 1.

FIG. 2 shows a disinfecting lighting device 101 according to another embodiment of the present invention. Analogously with the previous embodiment shown in FIG. 1, the disinfecting lighting device 101 comprises a housing 102 comprising an air disinfection cavity 103 delimited by a wall element 104. The air disinfection cavity 103 comprise a central portion 103′ and the peripheral portion 103″.

The wall element 104 comprises two transparent portions 105 and 105′ being substantially transparent to light from visible wavelength range. The disinfecting lighting device 101 comprises two disinfecting light sources 106, 106′ arranged inside the air disinfection cavity 103 and providing disinfecting UV light. The disinfecting light sources 106, 106′ are arranged in the peripheral portion 103′ of the air disinfection cavity 103. As may be seen in FIG. 2, the transparent portions 105 and 105′ comprise a reflective coating 109 for reflecting the disinfecting light.

The disinfecting lighting device 101 comprises two air inlet ducts 107 and two air outlet ducts 108 arranged in the wall element 104. The air inlet ducts 107 and the air outlet ducts 108 are positioned on the opposite sides of the wall element 102 in order to maximize the residing time of the air inside the air disinfection cavity 103.

As may be seen in FIG. 2, the air inlet ducts 107 and the air outlet ducts 108 are arranged such that the disinfecting light emitted by the disinfecting light sources 106, 106′ is prevented from escaping the air disinfection cavity 103 through the air inlet ducts 107 or the air outlet ducts 108 by introduction of a 90° turn in the air inlet ducts 107 and in the air outlet ducts 108.

In order to prevent the disinfecting light from escaping the air disinfection cavity 103, the transparent portions 105, 105′ comprise a reflective coating 109 for reflecting the disinfecting light. The reflective coating 109 completely covers the transparent portions 105 and 105′. The reflective coating 109 is arranged on the inner side of the transparent portions 105, 105′, i.e. on the side facing the air disinfection cavity 103.

The disinfecting lighting device 101 comprises two collimating optical elements 110, each arranged in the peripheral portion 103′ of the air disinfection cavity 103 for collimating the disinfecting light in a direction being parallel to the longitudinal extension of the transparent portions 105, 105′. The collimating optical elements 110 depicted in FIG. 2 are in the form of a concave reflective surface. According to such an embodiment, interactions between the disinfecting light and the transparent portions 105, 105′ is minimized or eliminated.

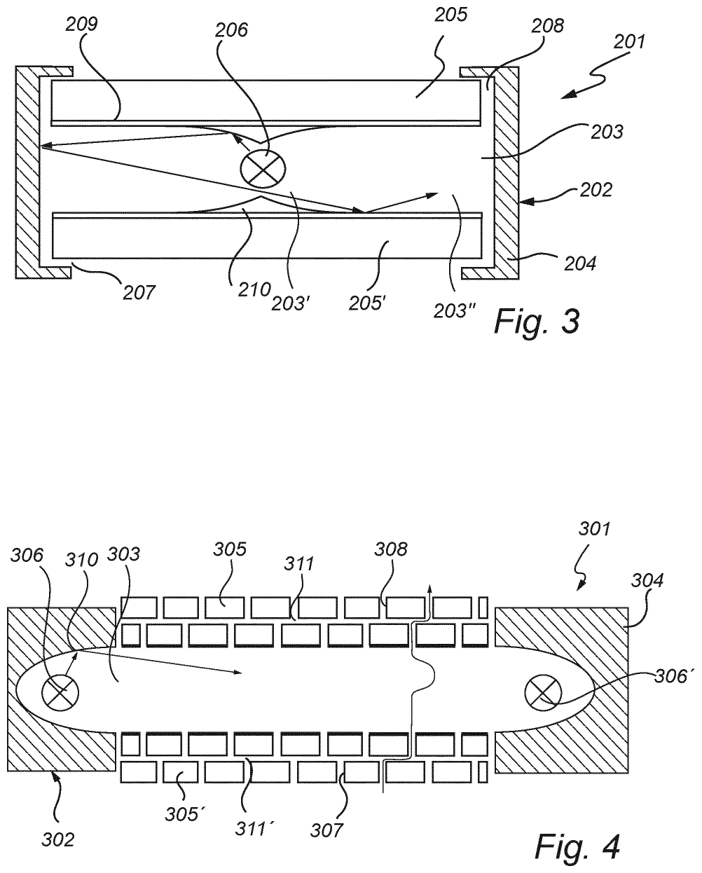

FIG. 3 shows another embodiment of the disinfecting lighting device according to the present invention. The disinfecting lighting device 201 is similar to the one shown in FIG. 2, thus the common features of these two embodiments will not be repeated. The disinfecting lighting device 201 comprises one disinfecting light source 206 arranged in the central portion 203′ of the air disinfection cavity 203. The collimating optical elements 210 are arranged for collimating the disinfecting light in a direction being parallel to the longitudinal extension of the transparent portions 205, 205′.

In FIG. 4, a disinfecting lighting device 301 is shown, having a similar structure as the disinfecting lighting device depicted in FIG. 2. As may be seen in FIG. 4, a plurality of air inlet ducts and a plurality of air outlet ducts are arranged in each of the transparent portions 305 and 305′. Each of the transparent portions 305 and 305′ comprise a first layer and a second layer, wherein each of the first layer and the second layer comprises a plurality of air inlet ducts and a plurality of air outlet ducts. The first and the second layer of each of the transparent portions 305 and 305′ are arranged adjacent to each other forming an intermediate air duct 311 and 311′. The plurality of air inlet ducts 307 in the first layer is offset relative the plurality of air inlet ducts in the second layer, and the plurality of air outlet ducts 308 in the first layer is offset relative the plurality of air outlet ducts in the second layer. The air ducts are perpendicular to the longitudinal extension of the transparent portions 305 and 305′ and to the intermediate air ducts 311 and 311′. The air to be disinfected passes through the plurality of air inlet ducts 307 in the first layer of the transparent portion 305′, moves through the intermediate air duct 311′ and continues to the air disinfection cavity 303 through the plurality of air inlet ducts 307 in the second layer of the transparent portion 305′. When the purified air exits the air disinfection cavity 303, it is first directed through the plurality of air outlet ducts 308 in the second layer of the transparent portion 305, moves through the intermediate air duct 311 and exits to the ambient through the plurality of air outlet ducts 308 in the first layer of the transparent portion 305. Such a configuration of the air inlet ducts and the air outlet ducts prevents the disinfecting light from escaping the air disinfection cavity through the air ducts, while providing highly efficient circulation of air through the disinfecting lighting device 301.

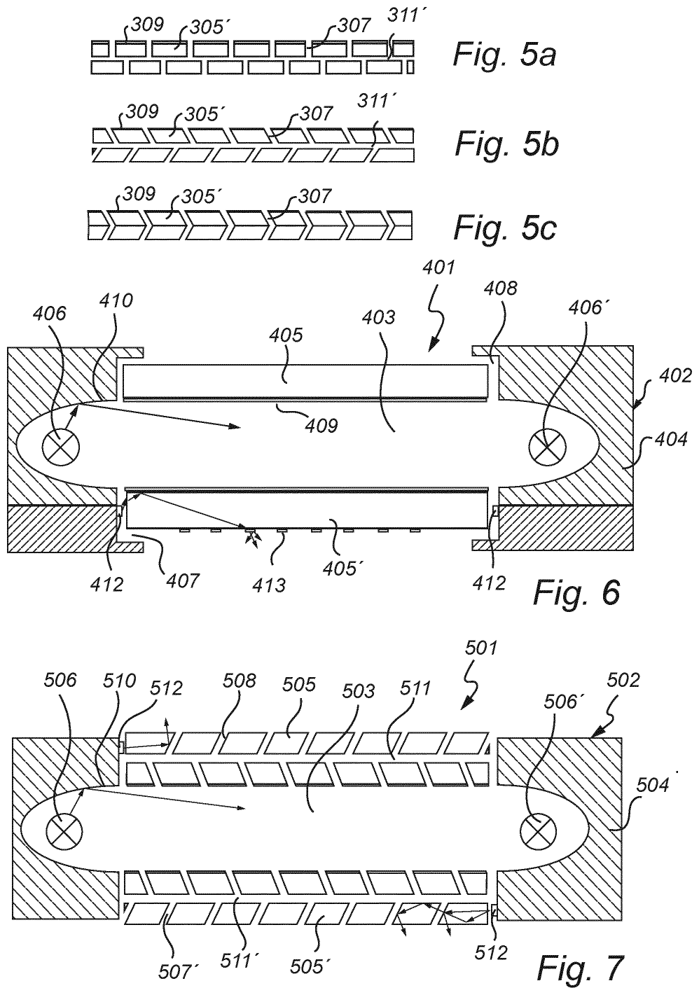

FIGS. 5a-5c illustrate different embodiments of arranging a plurality of air ducts in a transparent portion comprising two layers. As may be seen in FIG. 5b, the air ducts may be angled in relation to the longitudinal extension of the transparent portion 305′ and to the intermediate air duct 311′. Alternatively, the first and the second layers of the transparent portion 305′ may be arranged in direct contact with each other, i.e. without formation of an intermediate air duct as shown in FIG. 4c. In this case, the air ducts in the first layer of the transparent portion are aligned with the air ducts in the second layer, such that the air may pass through the layers. In such an embodiment, the air ducts in each of the layers are angled in relation to the longitudinal extension of the transparent portion 305′ in order to prevent the disinfecting light from escaping the air disinfection cavity.

In FIG. 6, the disinfecting lighting device 401 is shown, having a similar structure as the disinfecting lighting device depicted in FIG. 2. The disinfecting lighting device 401 comprise two non-disinfecting light sources 412 arranged to emit a non-disinfecting light, such as visible light. The non-disinfecting light sources 412 are arranged between the transparent portion 405′ and the wall element 404. The visible light will thus be emitted out of the transparent portion. As mentioned above, the visible light may have a signaling function indicating that the disinfecting light source is in operation, a decorative function such as glowing with tunable colours, a general lighting function or combinations thereof. In order to improve the performance of the disinfecting lighting device 401 by providing the visible light of high contrast, the transparent portion 405′ comprises a plurality of light extraction elements 413 arranged for extraction of the non-disinfecting light.

Turning the attention to FIG. 7, a disinfecting lighting device 501 is shown comprising two non-disinfecting light sources 512 each being arranged adjacent to the transparent portions 505 and 505′, respectively. The transparent portions 505 and 505′ function as a light guide for the non-disinfecting light. The air ducts 507, 508, 511 and 511′ function as light extractions elements.

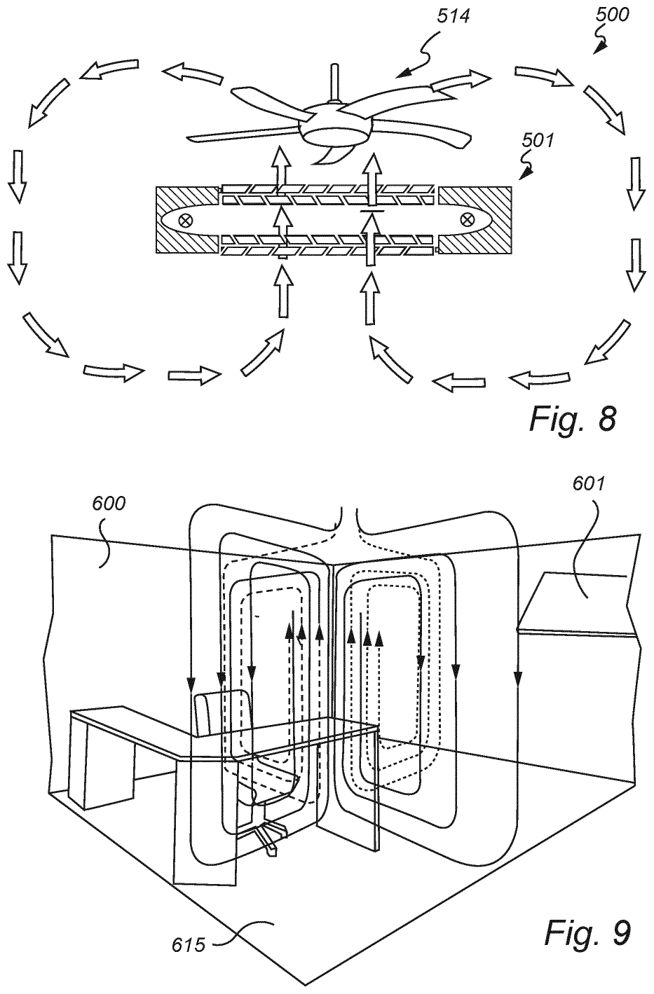

FIG. 8 depicts air disinfecting system 500 comprising a circulation means 514 in the form of a fan for generating a flow of air in a room, wherein the flow of air is directed in a flow direction. The air disinfecting system 500 further comprises the disinfecting lighting device 501 describe in detail above. As may be seen in FIG. 8, the plurality of air ducts is arranged substantially parallel to the flow direction. Alternatively, the circulation means 514 can be integrated into the air disinfection system 500.

FIG. 9 shows a room 600, wherein the disinfecting lighting device 601 is arranged on a wall. The disinfecting lighting device 601 is arranged in the air flow generated by the air vents in the room 600.

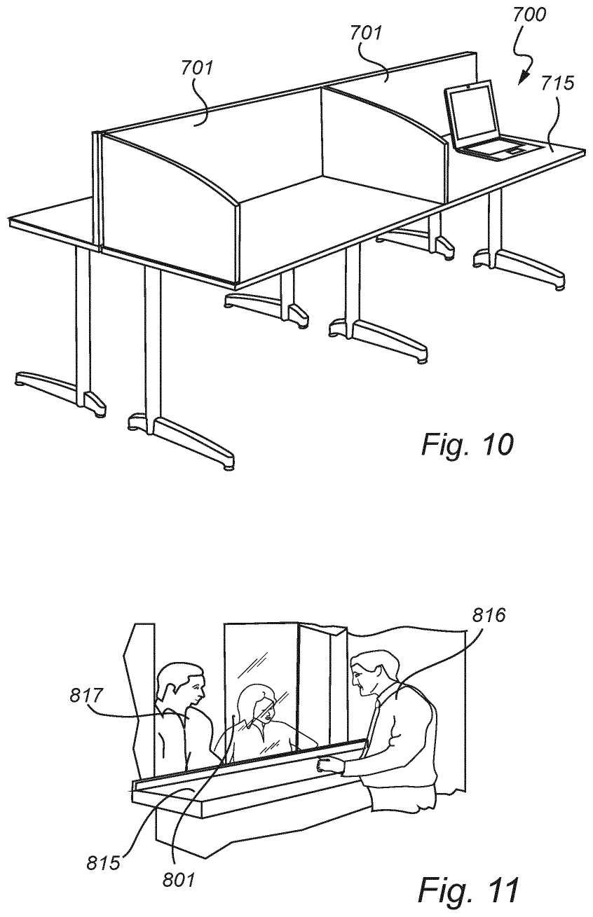

FIG. 10 illustrates another area of use for the disinfecting lighting device according to the present invention. In this embodiment, the disinfecting lighting device 701 is used as a desk divider in an open-plan office 700. The disinfecting lighting device 701 is arranged on the attachment surface 715, in this case being a desk. The two parallelly arranged transparent portions (not shown) are perpendicular to the attachment surface 715. The disinfecting lighting device 701 may function as a privacy window between the desks. Alternatively, or additionally, the disinfecting lighting device 701 may be used for providing desk light.

Yet another possible use of the disinfecting lighting device is depicted in FIG. 11. In this case, the disinfecting lighting device 801 is used as a counter shield between a clerk 816 and a customer 817. In analogy with the above, the disinfecting lighting device 801 is arranged on the attachment surface 815, in this case being a counter. The two parallelly arranged transparent portions (not shown) are perpendicular to the attachment surface 815.

Although the present invention has been described with reference to various embodiments, those skilled in the art will recognize that changes may be made without departing from the scope of the invention. It is intended that the detailed description be regarded as illustrative and that the appended claims including all the equivalents are intended to define the scope of the invention. While the present invention has been illustrated in the appended drawings and the foregoing description, such illustration is to be considered illustrative or exemplifying and not restrictive; the present invention is not limited to the disclosed embodiments. Other variations to the disclosed embodiments can be understood and effected by those skilled in the art in practicing the claimed invention, from a study of the drawings, the disclosure, and the appended claims. In the appended claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A disinfecting lighting device comprising:
a housing comprising an air disinfection cavity delimited by a wall element having a longitudinal extension and a transverse extension, said wall element comprising at least one transparent portion being substantially transparent to light from at least a portion of the visible wavelength range;
at least one disinfecting light source arranged inside said air disinfection cavity and providing disinfecting light;
at least one air inlet duct and at least one air outlet duct arranged in said wall element;
wherein said at least one transparent portion comprises a reflective coating for reflecting said disinfecting light, and
wherein said disinfecting lighting device further comprises at least one collimating optical element arranged for collimating said disinfecting light in a direction being parallel to said longitudinal extension of said at least one transparent portion.

2. The disinfecting lighting device according to claim 1, wherein said at least one transparent portion is transparent to visible light having wavelength in the range from 380 nm to 750 nm.

3. The disinfecting lighting device according to claim 1, wherein said reflective coating comprises a plurality of layers, and wherein at least one of said plurality of layers comprises HfO₂—SiO₂.

4. The disinfecting lighting device according to claim 1, wherein said at least one disinfecting light source is arranged for emitting at least one of: UV-C radiation, UV-B radiation, UV-A radiation, violet light and blue light.

5. The disinfecting lighting device according to claim 1, wherein said at least one air inlet duct and/or said at least one air outlet duct is angled in relation to said longitudinal extension of said wall element such that said disinfecting light is prevented from passing through at least one of said first air inlet duct and/or said at least one air outlet duct.

6. The disinfecting lighting device according to claim 1, wherein said at least one air inlet duct and/or said at least one air outlet duct is arranged in said at least one transparent portion.

7. The disinfecting lighting device according to claim 6, wherein said at least one transparent portion comprises a first layer and a second layer, wherein each of said first layer and said second layer comprises at least one air inlet duct and at least one air outlet duct, and wherein said first and said second layer are arranged adjacent to each other forming an intermediate air duct such that said at least one air inlet duct in said first layer is offset relative said at least one air inlet duct in said second layer, and said at least air outlet duct in said first layer is offset relative said at least one air outlet duct in said second layer.

8. The disinfecting lighting device according to claim 1, wherein said disinfecting lighting device further comprises at least one non-disinfecting light source arranged to emit a non-disinfecting light.

9. The disinfecting lighting device according to claim 8, wherein said at least one non-disinfecting light source is arranged adjacent to said at least one transparent portion, and wherein said at least one transparent portion comprises at least one light extraction element arranged for extraction of said non-disinfecting light.

10. The disinfecting lighting device according to claim 8, wherein said at least one non-disinfecting light source is electrically coupled to said disinfecting light source.

11. The disinfecting lighting device according to claim 1, wherein said air disinfection cavity comprises a central portion and a peripheral portion, and wherein said disinfecting lighting device comprises two disinfecting light sources being arranged opposite to each other in said peripheral portion of the air disinfection cavity.

12. The disinfecting lighting device according to claim 1, wherein said wall element comprises two parallelly arranged transparent portions.

13. The disinfecting lighting device according to claim 12, wherein said disinfecting lighting device is arranged on an attachment surface such that said two parallelly arranged transparent portions are perpendicular to said attachment surface.

14. An air disinfecting system comprising a circulation means for generating a flow of air in a room, said flow of air being directed in a flow direction, wherein said air disinfecting system comprises the disinfecting lighting device according to claim 1, and wherein said at least one air inlet duct and/or at least one air outlet duct is arranged parallel to said flow direction.
